(19) United States
(12) Patent Application Publication
(10) Pub. No.: US 2002/0187944 A1
(43) Pub. Date: Dec. 12, 2002

(54) Inhibition of human immunodeficiency virus reverse transcriptase

(76) Inventor: Paul Porwen Hung, Bryn Mawr, PA (US)

Correspondence Address: Y. ROCKY TSAO, Fish & Richardson P.C., 225 Franklin Street, Boston, MA, 02110-2804, US

(21) Appl. No.: 10/103,201

(22) Filed: Mar. 21, 2002

Related U.S. Application Data
(60) Provisional application No. 60/277,583, filed on Mar. 21, 2001.

(51) Int. Cl. A61K 31/7048

(52) U.S. Cl. 514/29

(57) ABSTRACT

A method of inhibiting a human immunodeficiency virus reverse transcriptase by contacting the reverse transcriptase with an effective amount of 9-O-methyl oxime erythromycin A.

### RELATED APPLICATION

This application claims priority from U.S. Provisional Patent Application Serial No. 60/277,583, filed Mar. 21, 2001.

### BACKGROUND OF THE INVENTION

Human immunodeficiency virus (HIV) has proven to be an intractable pathogen. One strategy has been to target the reverse transcriptase of this virus.

### SUMMARY OF THE INVENTION

The invention is based on the unexpected discovery that 9-O-methyl oxime erythromycin A inhibits HIV reverse transcriptase and thereby blocks HIV replication at low concentrations, as compared with previous studies on other retroviruses.

9-O-methyl oxime erythromycin A, as used herein, includes the compound itself, as well as its salts and prodrugs, if applicable. Such salts can be formed by interaction between a positively charged substituent (e.g., amine) on the 9-O-methyl oxime erythromycin A molecule with a negatively charged counterion. Suitable counterions include, but are not limited to, chloride, bromide, iodide, sulfate, nitrate, phosphate, and acetate. Examples of prodrugs include esters and other pharmaceutically acceptable derivatives, which, upon administration to a subject, are converted to 9-O-methyl oxime erythromycin A. Accordingly, in one aspect, the invention features a method of inhibiting an HIV reverse transcriptase by contacting the reverse transcriptase with 9-O-methyl oxime erythromycin A in an effective amount. In other words, contemplated within the scope of this invention is a method of treating a patient infected with HIV by administering to the patient an effective amount of 9-O-methyl oxime erythromycin A. The term “treating” is defined as the administration of a composition including 9-O-methyl oxime erythromycin A to a subject, who has HIV infection, a symptom of HIV infection, a disease state secondary to HIV infection, or a predisposition toward HIV infection, with the purpose to cure, alleviate, relieve, remedy, or ameliorate the infection, the symptom of the infection, the disease state secondary to the infection, or the predisposition toward the infection. “An effective amount” is the amount of 9-O-methyl oxime erythromycin A which, upon administration to a subject infected by HIV, is required to inhibit HIV reverse transcriptase and confer therapeutic effect on the subject. An effective amount of 9-O-methyl oxime erythromycin A may range from 0.001 mg/Kg to 100 mg/Kg. Effective doses vary, as recognized by those skilled in the art, depending on route of administration, excipient usage, and the possibility of co-usage with other agents for treating HIV infection.

The invention also covers a pharmaceutical composition containing 9-O-methyl oxime erythromycin A as an active anti-HIV agent, as well as use of 9-O-methyl oxime erythromycin A for the manufacture of a medicament for treating HIV infection.

Also within the scope of this invention is a packaged product. The packaged product includes a container, a pharmaceutical composition including 9-O-methyl oxime erythromycin A as an active anti-HIV agent in the container, and a legend (e.g., a label or an insert) associated with the container and indicating administration of the pharmaceutical composition for treating HIV infection (e.g., AIDS, or HIV infection-associated diseases such as Kaporsi' sarcoma and pneumocystis cariini).

Other features or advantages of the present invention will be apparent from the following detailed description, and also from the claims.

### DETAILED DESCRIPTION OF THE INVENTION

The invention relates to a method of inhibiting HIV reverse transcriptase by contacting the reverse transcriptase with an effective amount of 9-O-methyl oxime erythromycin A.

9-O-methyl oxime erythromycin A can be prepared by procedures well known to a skilled person in the art. See, e.g., U.S. Pat. No. 3,681,326. One synthetic route follows: An acid (e.g., hydrochloride) is pre-mixed with methyl hydroxylamine to form an acid addition salt. The salt is then added to an alcohol (e.g., ethanol) that contains erythromycin A. The reaction mixture is heated at a temperature of from about 20° C. to about the reflux temperature of the alcohol. After the reaction is complete, the mixture is cooled down. The 9-O-methyl oxime erythromycin A thus obtained can be further purified by column chromatography, high pressure liquid chromatography, recrystallization, or other suitable methods. The starting material, erythromycin A, can be produced in a culture of Streptomyces erythreus (Waksman) Waksman & Henrici, and purified from the culture. See, e.g., U.S. Pat. Nos. 2,653,899 and 2,823,203. Alternatively, erythromycin A can be synthesized either from organic chemicals (Woodward et al. (1981) J. Am. Chem. Soc. 103:3215) or from biological materials (Martin et al. (1975) Tetrahedron 31:1985). In addition to the just-described synthetic route, various synthetic steps may be performed in an alternate order to give the desired 9-O-methyl oxime erythromycin A. Synthetic chemistry transformations and protecting group methodologies useful in synthesizing 9-O-methyl oxime erythromycin A are known in the art and include, for example, those described in Larock (1989) Comprehensive Organic Transformations, VCH Publishers; Greene and Wuts (1991) Protective Groups in Organic Synthesis, 2d. Ed., John Wiley and Sons; and Paquette, ed. (1995) Encyclopedia of Reagents for Organic Synthesis, John Wiley and Sons.

9-O-methyl oxime erythromycin A can be administered orally or parenterally for in vivo inhibition of HIV reverse transcriptase. See, e.g., U.S. Pat. No. 3,979,511; Huang et al. (1991) J. of Med. Virol. 35:180-186; and Huang (1975) J. Gen. Virol. 26:135-139. The term “parenteral” as used herein includes subcutaneous, intracutaneous, intravenous, intramuscular, intraarticular, intraarterial, intrasynovial, intrasternal, intrathecal, intralesional and intracranial injection, as well as various infusion techniques. 9-O-methyl oxime erythromycin A is a derivative of erythromycin A, an antibacterial drug. Erythromycin A is usually formulated as tablets for oral administration. Since it is acid labile, its prodrugs such as ethyl succinate and stearate are used for protection from gastric acid. 9-O-methyl oxime erythromycin A, on the other hand, is acid stable. Given its chemical similarity to erythromycin A, it can also be orally administered as tablets. 9-O-methyl oxime erythromycin A has also been known to be dissolved in dimethyl sulfoxide (DMSO) and injected to a subject.

A composition for oral administration can be any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, 9-O-methyl oxime erythromycin A can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

Without further elaboration, it is believed that one skilled in the art can, based on the above disclosure and the example below, utilize the present invention to its fullest extent. The following example is to be construed as merely illustrative, and is not limitative of the remainder of the disclosure in any way. All publications cited in this disclosure are hereby incorporated by reference.

Inhibition of HIV reverse transcriptase (RT) was studied in a one milliliter reaction mixture containing 12.5 μmol Tris-HCl (pH 8.3), 3 μmol MgCl₂, 10 μmol KCl, 2.5 μmol dithiothreitol, 0.04 μmol of each dNTP, 1 μCi ³H-TTP (50 Ci/mmol), 10 μl of DMSO containing 0-320 μg/ml of 9-O-methyl oxime erythromycin A (EMO), and 0.01 μg HIV-1 reverse transcriptase. DNA template (0.01 μg) from calf thymus was used as the substrate in the reaction. After 60 minutes incubation at 37° C., samples from the reaction were pipetted on 3 MM filter paper discs (Whatman), which were washed 5 times in cold trichloroacetic acid (5%) containing sodium pyrophosphate (0.01 M). After drying, the discs were counted in a toluene scintillation solution using a Beckman scintillation spectrometer. Reactions with DNA-dependent DNA polymerase (DNA pol I, GibcoBRL) or DNA-dependent RNA polymerase from E. coli were carried out as described above, except that ³H-UTP replaced ³H-TTP where appropriate. The results are shown in the table below:

| | | | DNA-dependent DNA | | DNA-dependent RNA | |
|---|---|---|---|---|---|---|
| | HIV RT | | polymerase | | polymerase | |
| [EMO] | Radioactivity | | Radioactivity | | Radioactivity | |
| (μg/ml) | (cpm) | % Inhibition | (cpm) | % Inhibition | (cpm) | % Inhibition |
| 0 | 8560 | 0 | 12,500 | 0 | 32,500 | 0 |
| 20 | 8490 | 0.8 | N/D | N/D | N/D | N/D |
| 40 | 8600 | −0.5 | N/D | N/D | N/D | N/D |
| 80 | 6850 | 20 | 12,800 | −2 | 33,000 | −0.2 |
| 160 | 1625 | 81 | 12,400 | 1 | 32,800 | −1 |
| 320 | 1287 | 85 | 12,600 | 0 | 32,400 | 0.5 |

As shown in the above table, 9-O-methyl oxime erythromycin A unexpectedly inhibited HIV RT by 81% at 160 μg/ml.

### Other Embodiments

It is to be understood that while the invention has been described in conjunction with the detailed description thereof, the foregoing description is intended to illustrate and not limit the scope of the invention, which is defined by the appended claims. Other aspects, advantages, and modifications are within the scope of this invention.

What is claimed is:

1. A method of inhibiting a human immunodeficiency virus reverse transcriptase, the method comprising contacting the reverse transcriptase with an effective amount of 9-O-methyl oxime erythromycin A.
